(19) United States
(12) Patent Application Publication
(10) Pub. No.: US 2021/0100674 A1
(43) Pub. Date: Apr. 8, 2021

(54) Posture Correcting Back Brace

(71) Applicant: Ezequiel C. Tzintzun, JR., Harrah, WA (US)

(72) Inventor: Ezequiel C. Tzintzun, JR., Harrah, WA (US)

(21) Appl. No.: 16/591,567

(22) Filed: Oct. 2, 2019

(51) Int. Cl. A61F 5/02

(52) U.S. Cl. CPC A61F 5/026

(57) ABSTRACT

A posture correcting back brace device that provides pressure and support to the spinal and lumbar areas of the user, with an inverted “T” shaped plate having a substantially rigid half-round bar running vertically, approximately positioned along the user's spine, and a substantially rigid half-round bar running horizontally, along the waist of the user proximate the lumbar area. The T-plate attaches to a harness with a belt-loop and a pair of arm-loops, fitted to the user, with the waist bar fitting approximately to a lordotic curvature of a lumbar region of the user, and the spinal bar approximately following the spine of the user, upward approximately to the user's upper back.

### TECHNICAL FIELD

A Posture Correcting Back Brace that provides pressure and support to the spinal and lumbar areas of the user, and specifically a wearable brace that includes an inverted “T” shaped plate, having a half-round bar running vertically, approximately positioned along the user's spine, and having a half-round bar running horizontally, along the waist of the user proximate the lumbar area.

### BACKGROUND OF THE INVENTION

Serious and painful back problems, many requiring corrective surgery, have spawned a multitude of devices to provide support and relief to specific regions of the back. However, few known devices provide effective support to both the spine and the lumbar areas, and so there is a need for a device that better serves to maintain the normal alignment of the spine, while providing supportive pressure on the muscles of the lower back. Also, such a device should be simple to fit and comfortable to wear, and provide needed support and pressure on the back and lumbar areas of the wearer.

The following is a disclosure of the present invention that will be understood by reference to the following detailed description taken in conjunction with the accompanying drawings.

### BRIEF DESCRIPTION OF DRAWINGS

FIG. 1 is a front view of a posture correcting back brace, according to an embodiment of the invention;

FIG. 2 is a back view of a posture correcting back brace, as used according to an embodiment of the invention;

FIG. 3 is a back view of a posture correcting back brace attached to a user, according to an embodiment of the invention;

FIG. 4 is a front view of a posture correcting back brace attached to a user, according to an embodiment of the invention; and

FIG. 5 is a perspective view of a posture correcting back brace, according to an embodiment of the invention.

Reference characters included in the above drawings indicate corresponding parts throughout the several views, as discussed herein. The description herein illustrates one preferred embodiment of the invention, in one form, and the description herein is not to be construed as limiting the scope of the invention in any manner. It should be understood that the above listed figures are not necessarily to scale and that the embodiments are sometimes illustrated by fragmentary views, graphic symbols, diagrammatic or schematic representations, and phantom lines. Details that are not necessary for an understanding of the present invention by one skilled in the technology of the invention, or render other details difficult to perceive, may have been omitted.

### DETAILED DESCRIPTION OF SPECIFIC EMBODIMENTS

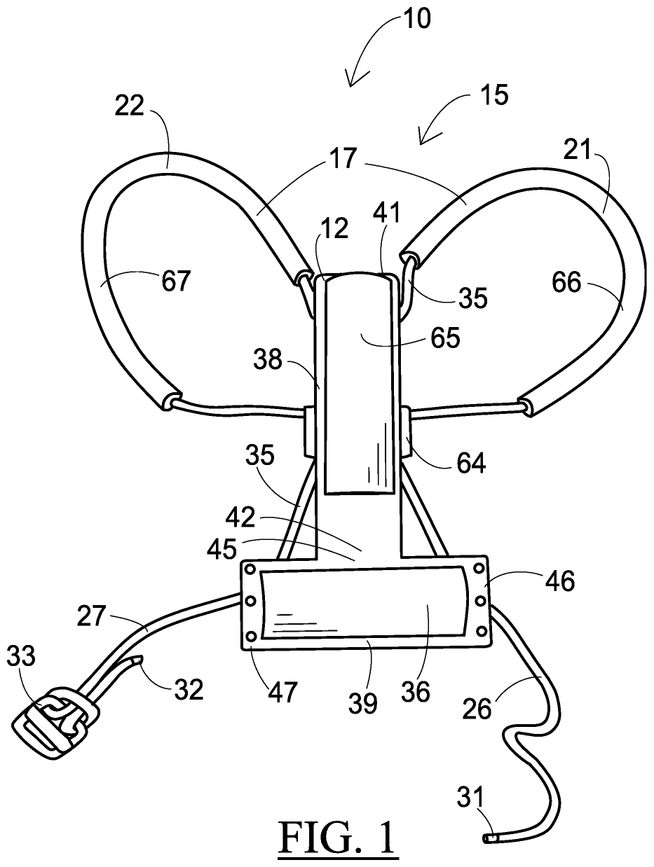

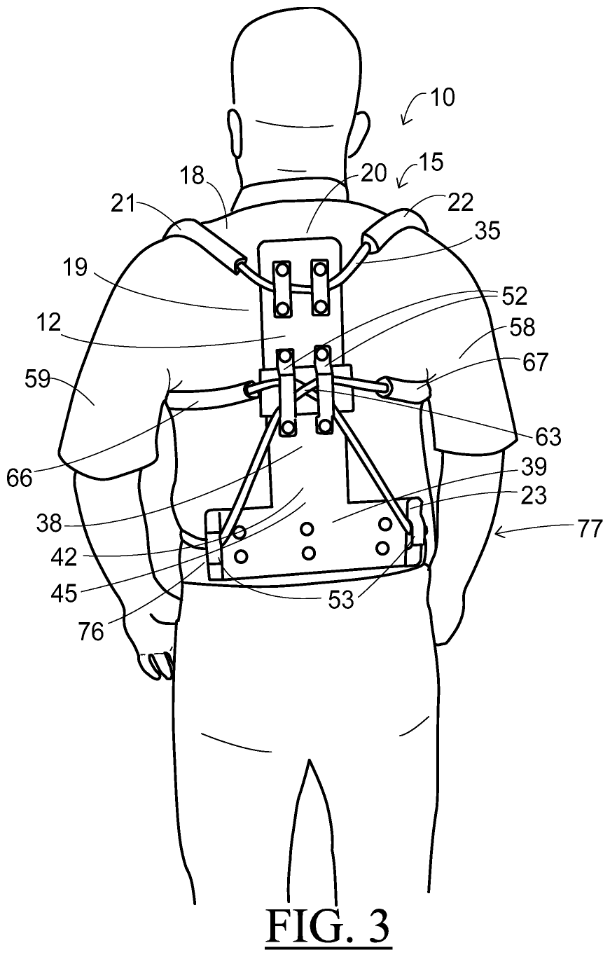

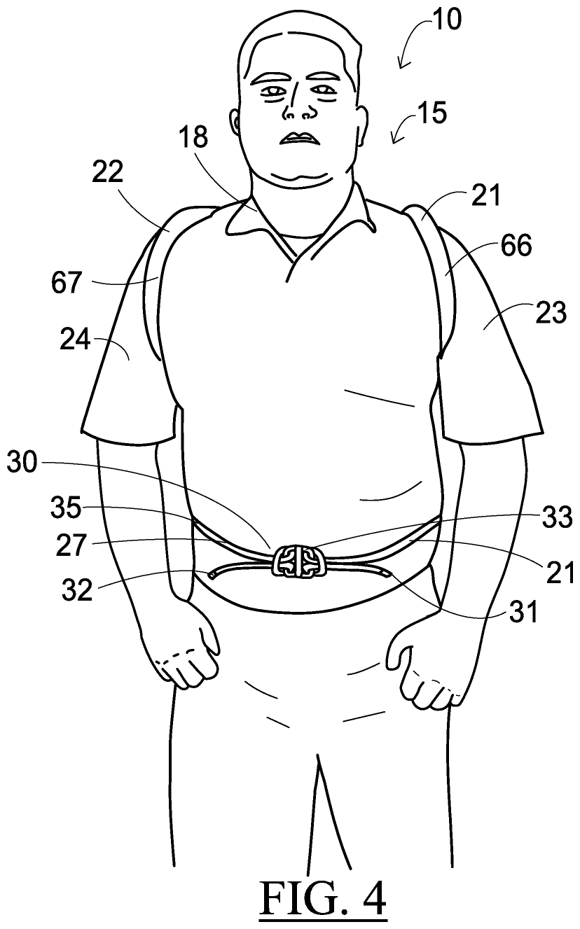

The invention provides a posture correcting back brace device. Alternatively, the posture correcting back brace device of the present invention may be referred to herein as a ‘back brace device,’ or simply as the ‘Back Brace.’ The Back Brace 10 is shown in FIGS. 1 through 5, and the Back Brace includes a T-plate 12, attached to a harness 15, as shown in FIG. 1. The harness includes a belt-loop 16 and a pair of arm-loops 17 that are fitted to a user 18, as shown in FIGS. 3 and 4.

The pair of arm-loops 17 of the harness include a first arm-loop 21 that receives a first arm of the user, and a second arm-loop 22, that receives a second arm of the user, with the user 18 fitting into harness of the Back Brace 10 by placing a first arm 23 into the first arm-loop and a second arm 24 into the second arm-loop of the harness, as shown in FIG. 4. The spine plate fits to the user proximate to an upper back 19 of the user, as shown in FIG. 3. Additionally, the belt-loop 16 includes a first belt-segment 26 and a second belt-segment 27, with the first belt-segment connectable to the second belt-segment around the waist 30 of the user, as shown in FIG. 4.

Preferably, the first belt-segment terminates with a first belt-end 31, and the second belt-segment terminates with a second belt-end 32, with the second belt-end threaded into a buckle 33. Most preferably, the buckle is a standard ‘loop through’ type of buckle, as shown in FIG. 1, with the first belt-end 31 threadable into the buckle and pullable by the user 18 to tighten the belt around the waist 30 of the user, as shown in FIG. 4. Alternatively, the buckle can be any type of buckle that is known those skilled in buckle design or manufacture.

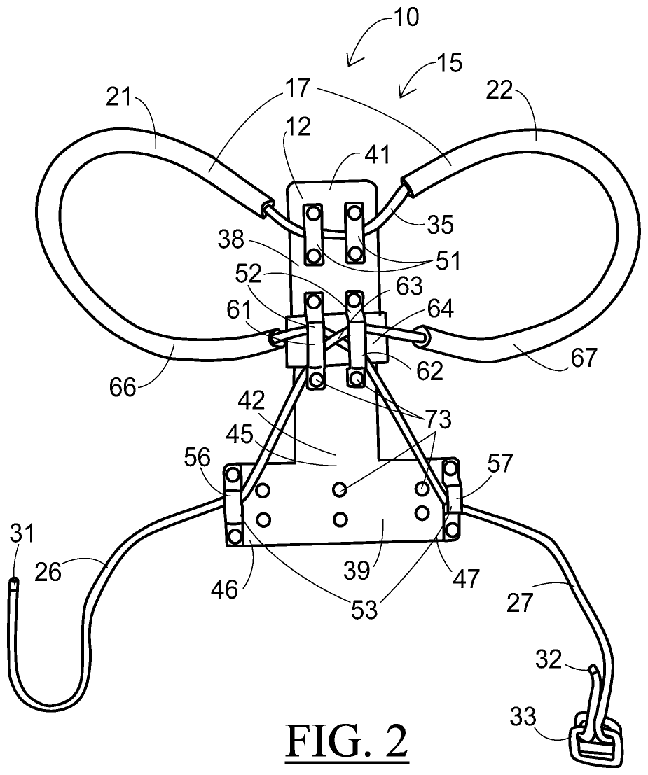

Also most preferably, the belt-loop 16 and the arm-loops 17 of the harness 15 are a continuous length of a harness rope 35, as shown in FIG. 2. For this most preferred embodiment of the Back Brace 10, the continuous harness rope begins at the first belt-end 31, and terminates at the second belt-end 32. In this most preferable embodiment of the Back Brace, the harness rope is most preferably a ‘kern mantle’ type of rope, as often employed for boating ropes and for climbing ropes, having a rope core sleeved with a woven outer sheath as well known to those skilled in rope construction. The core fibers of the harness rope provide substantially all of the tensile strength of the rope, while the sheath protects the core from abrasion and cutting during use. A preferred diameter of the harness rope is approximately 11 mm (0.43 inches).

As shown in FIG. 2, the T-plate 12 is preferably in the approximate shape of an inverted “T,” when worn by the user 18, proximate to a back side or “back” 19 of the user, with a spine plate 38 intersecting a waist plate 39. When the Back Brace 10 is worn on the user, the spine plate is substantially vertical, and the waist plate is substantially horizontal, as shown in FIG. 3. Additionally, the spine plate terminates upwardly at a plate top 41 and terminates downwardly at a plate base 42. The waist plate extends from the plate base at a plate intersection 45, with the waist plate extending horizontally and terminating at each end, at a first waist plate end 46 and a second waist plate end 47.

Preferably, the T-plate 12 is a single piece of resilient plastic material that is bendable without breaking, yet provides a substantially rigid framework for the Back Brace. A most preferred material for the T-plate is a high density, high molecular weight polyethylene (HDPE). Polyethylene is most preferred because it is moderately flexible, yet at the preferred thickness of approximately 0.1 inches (2.5 mm), the T-plate made of HDPE is resilient and resistant to cracking or fracture and chemical or sunlight degradation. Any material that provides the desired properties of flexibility without breaking or fracture could be employed for the T-plate, including a metal, plastic or wood material, as could be selected by persons skilled in the engineering and selection of resilient materials.

Also preferably, the T-plate 12 includes a top-rope bracket 51, a mid-rope bracket 52 and a bottom-rope bracket 53, with the bottom rope bracket most preferably including a first bottom-rope bracket 56 mounted proximate to the first waist plate end 46, and a second bottom-rope bracket 57 mounted proximate to the second waist plate end 47, as shown in FIGS. 2 and 3. The top-rope bracket, the mid rope bracket and the bottom-rope bracket receive the harness rope, most preferably routed as shown in FIG. 2.

In a preferred embodiment of the Back Brace 10, the harness rope 35 threads through the T-plate 12, with the first end belt-end 31 threaded through the second bottom-rope bracket 57, then though the mid-rope bracket 52, and then to the top-rope bracket 51. This first loop of harness rope formed between the mid-rope bracket and the top-rope bracket is the first arm-loop 21 of the harness 15, and receives a first arm 58 of the user 18, as shown in FIG. 3.

After forming the first arm-loop 21, from top-rope bracket 51, the harness rope 35 then threads again through the T-plate 12, with the first belt-end 31 of the harness rope then re-threaded through the mid-rope bracket 52. This second loop of harness rope formed between the mid-rope bracket and the top-rope bracket is the second arm-loop 22 of the harness 15, and receives a second arm 59 of the user 18, as shown in FIG. 3.

After forming the second arm-loop 22, from mid-rope bracket 52, the harness rope 35 then threads again through the T-plate 12, with the first belt-end 31 of the harness rope then threads through the second bottom-rope bracket 57.

Also preferably, the mid-rope bracket 52 includes a first mid-rope bracket 61 and a second mid-rope bracket 62, as shown in FIG. 2, which provides for a rope crossing 63 of the harness rope 35. The rope crossing allows the harness rope to route from the second arm-loop 22 of the harness 15 to the second bottom-rope bracket 53, and provides for friction in the harness rope routing throughout the harness. Additionally, as shown in FIG. 2, a friction plate 64 can be mounted between the T-plate 12 and the mid-rope bracket to increase resistance of the harness rope within the harness.

The remainder of the harness rope 35 extending from the second bottom-rope bracket to form the second belt-segment 27, and the remainder of the harness rope extending from the first bottom-rope bracket 56 to form the first belt-segment 26. The first belt-segment 26 and the second belt segment together form the belt-loop 16, with the first belt-segment connectable to the second belt-segment around the waist 30 of the user 18, as shown in FIG. 4

Preferably, the first arm-loop 21 includes a first arm-loop pad 66 and the second arm-loop 22 includes a second arm-loop pad 67. The first arm-loop pad and the second arm-loop pad are most preferably a closed cell type of foam, commonly employed as pipe insulation, and selected to fit the diameter of the harness rope 35 and provides cushioning to the user 18. Specifically, the first arm-loop pad cushions the user proximate to the first arm 61 of the user, and the second arm-loop pad cushions the user proximate to the second arm 62 of the user, as shown in FIGS. 3 and 4.

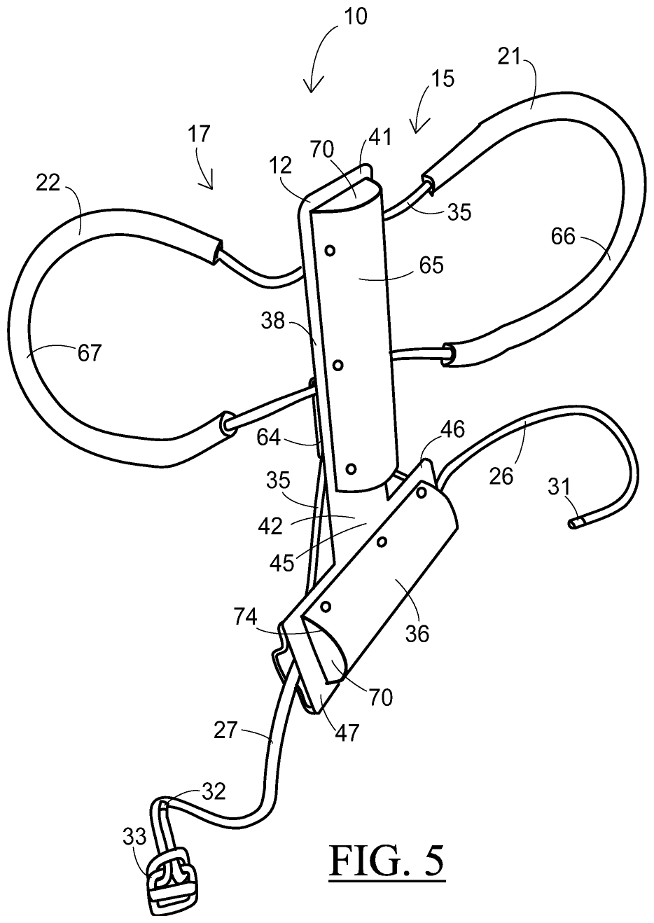

FIG. 1 shows a preferred embodiment of the Back Brace 10, from the front, with the T-plate 12 of the Back Brace including a spinal bar 65 mounted to the spine plate 38 of the T-plate, and a waist bar 66 mounted to the waist plate 38 of the T-plate. The spinal bar and the waist bar are substantially half-round in form, and both curve away from the T-plate and toward the user 18 when worn by the user, with approximately a hemispherical cross section. Most preferably, the spinal bar and the waist bar are both substantially rigid in construction, and formed from a standard metal pipe that is cut in half, lengthwise. A most preferable diameter for the spinal bar and the waist bar is approximately 4 inches (10.2 cm), but may range from 3 inches (7.6 cm) to 6 inches (15.2 cm). A most preferably pipe for the spinal bar and the waist bar is aluminum irrigation pipe, typically having a wall thickness of approximately 0.05 inches (1.27 mm). In the preferred form of a half-pipe, the spinal bar is preferably approximately 18 inches (45.7 cm) in length, and mounted to the spine plate of the T-plate between the plate top 41 and the plate intersection 45, as shown in FIG. 1. Also in the preferred form of a half-pipe, the waist bar is preferably approximately 12 inches (30.5 cm) in length, and mounted to the waist plate of the T-plate, between the first waist plate end 46 and the second waist plate end 47, also as shown in FIG. 5.

The spinal bar 65 and the waist bar 66 are preferably formed from a light and easily machined metal, such as an aluminum alloy, as discussed above. An aircraft grade of aluminum alloy could be employed. Other materials including metals, composites and plastics are considered for forming the bars, such as a titanium, a carbon fiber, or a high impact poly-vinyl-chloride (PVC) as typified by “Vintec” brand of Type II, PVC.

Specifically, in a preferred embodiment of the Back Brace 10, a spinal bar 65 and waist bar 66, are hollow, as discussed above to reduce the weight of the Back Brace, with the spinal bar and the waist bar each capped with a core 70, as shown in FIG. 5. Most preferably, the spinal core mounts to the T-plate 12, with a multiple of spinal core mounts 72, which are most preferably screws, and with the spinal bar then attaches to the spinal core with an adhesive 74. Likewise, the waist core mounts to the T-plate, with a multiple of waist core mounts 73, which are most preferably screws, and with the waist bar then attaches to the waist core with the adhesive. The adhesive can be any conventional adhesive, preferably water proof and permanent, selectable by those skilled in adhesive, to adhere the materials of the selected spinal core to the materials of the selected spinal bar, and to adhere the waist core to the materials of the selected waist bar.

Most preferably, the Back Brace 10, is light in weight at approximately 3 pounds (1.4 kg) to 5 pounds (2.3 kg), to minimize strain on the user 18 while attached to and carrying the Back Brace. Also, when worn by the user as shown in FIG. 3, the Back Brace, fits comfortably and fits approximately anatomically to the user. Specifically, the waist bar 66 fits approximately to the normal, inward lordotic curvature 76 of the lumbar region 77 of the user, and then the spinal bar 65 approximately follows the spine 20 of the user upward, approximately to the upper back 19 of the user, as also shown in FIG. 3.

Of note, the terms “substantially,” “proximate to” and “approximately” are employed herein throughout, including this detailed description and the attached claims, with the understanding that is denotes a level of exactitude or equivalence in amount or location commensurate with the skill and precision typical for the particular field of endeavor, as applicable.

In compliance with the statutes, the invention has been described in language more or less specific as to structural features and process steps. While this invention is susceptible to embodiment in different forms, the specification illustrates preferred embodiments of the invention with the understanding that the present disclosure is to be considered an exemplification of the principles of the invention, and the disclosure is not intended to limit the invention to the particular embodiments described. Those with ordinary skill in the art will appreciate that other embodiments and variations of the invention are possible, which employ the same inventive concepts as described above. Therefore, the invention is not to be limited except by the following claims, as appropriately interpreted in accordance with the doctrine of equivalents.

The following is claimed:

1. A back brace device comprising:
a T-plate attached to a harness, the T-plate including a spine plate and a waist plate;
a harness including a belt-loop attached to the waist plate and a pair of arm-loops attached to the spine plate, the belt-loop and the pair of arm-loops fitted to a user;
the pair of arm-loops of the harness having a first arm-loop and a second arm-loop, with the user fitting into the harness by the user placing a first arm into the first arm-loop, the user placing a second arm into the second arm-loop of the harness, and the belt-loop secured around the waist of the user;
the T-plate of the back brace including a spinal bar mounted to the spine plate of the T-plate, and a waist bar mounted to the waist plate of the T-plate, with the spinal bar substantially half-round in form and curving away from the T-plate, and the waist bar substantially half-round in form and curving away from the T-plate;
the spinal bar substantially rigid, and the waist bar substantially rigid; and
the back brace fitted to the user, with the waist bar fitting approximately to a lordotic curvature of a lumbar region of the user, and then the spinal bar approximately follows a spine of the user upward, approximately upward along an upper back of the user.

2. The back brace device of claim 1, wherein:
the belt-loop includes a first belt-segment and a second belt-segment, with the first belt-segment connectable to the second belt-segment around a waist of the user, the first belt-segment terminating with a first belt-end, and the second belt-segment terminates with a second belt-end, with the second belt-end threaded into a buckle.

3. The back brace device of claim 1, wherein the belt-loop and the pair of arm-loops of the harness are a continuous length of a harness rope.

4. The back brace device of claim 1, wherein:
the T-plate is a single piece of resilient plastic material, and resilient plastic material that is bendable without breaking, and the T-plate forms a substantially rigid framework for the back brace device.

5. A back brace device comprising:
a T-plate attached to a harness, the T-plate including a spine plate and a waist plate;
the harness including a belt-loop and a pair of arm-loops;
the pair of arm-loops of the harness having a first arm-loop and a second arm-loop;
the T-plate including a spinal bar mounted to the spine plate of the T-plate, and a waist bar mounted to the waist plate of the T-plate, with the spinal bar substantially half-round in form and curving away from the T-plate, and the waist bar substantially half-round in form and curving away from the T-plate;
the spinal bar substantially rigid, and the waist bar substantially rigid; and
the back brace fit-able approximately anatomically to a user, with the first arm of the user receivable into the first arm-loop and the second arm of the user receivable into the second arm-loop, with the spine plate attachable to the back of the user and the spinal bar approximately follows a spine of the user upward, approximately to a shoulder area of the user, and with the waist plate attached to the waist of the user with the belt loop, and the waist bar receivable approximately onto a lordotic curvature of a lumbar region of the user,

6. The back brace device of claim 5, wherein:
the pair of arm-loops of the harness includes a first arm-loop and a second arm-loop; and
the user fits into the harness by the user placing a first arm into the first arm-loop, and by the user placing a second arm into the second arm-loop of the harness.

7. The back brace device of claim 5, wherein:
the belt-loop includes a first belt-segment and a second belt-segment, with the first belt-segment connectable to the second belt-segment around a waist of the user, the first belt-segment terminating with a first belt-end, and the second belt-segment terminates with a second belt-end, with the second belt-end threaded into a buckle.

8. The back brace device of claim 5, wherein:
the belt-loop and the pair of arm-loops of the harness are a continuous length of a harness rope.

9. The back brace device of claim 5, wherein:
the T-plate is a single piece of resilient plastic material, and resilient plastic material that is bendable without breaking, and the T-plate forming a substantially rigid framework for the back brace device.

10. A back brace device comprising:
a T-plate attached to a harness, the T-plate having an approximate shape of an inverted “T” and the T-plate including a spine plate and a waist plate;
the spine plate terminating upwardly at a plate top and the spine plate terminating downwardly at a plate base, and the waist plate extending from the plate base at a plate intersection, with the waist plate extending horizontally from the connection to the spine plate at the plate intersection, and with the waist plate terminating at a first waist plate end and a second waist plate end;
the harness including a belt-loop and a pair of arm-loops, the belt loop attached to the waist plate, and the belt loop attached to the spine plate;
the pair of arm-loops of the harness having a first arm-loop and a second arm-loop;
the T-plate including a spinal bar mounted to the spine plate of the T-plate, and a waist bar mounted to the waist plate of the T-plate, with the spinal bar substantially half-round in form and curving away from the T-plate, and the waist bar substantially half-round in form and curving away from the T-plate;
the back brace fit-able to a user, the user having a first arm, a second arm, a lumbar region, a spine, and a waist; and
the first arm of the user receivable into the first arm-loop, the second arm of the user receivable into the second arm-loop, the
the waist bar receivable approximately onto a lordotic curvature of the lumbar region of the user, and the spinal bar approximately follows the spine of the user upward, approximately to an upper back area of the user.

11. The back brace device of claim 10, wherein the spinal bar is substantially rigid.

12. The back brace device of claim 10, wherein the waist bar is substantially rigid.

13. The back brace device of claim 10, wherein:
the belt-loop includes a first belt-segment and a second belt-segment, with the first belt-segment connectable to the second belt-segment around a waist of the user, the first belt-segment terminating with a first belt-end, and the second belt-segment terminates with a second belt-end, with the second belt-end threaded into a buckle.

14. The back brace device of claim 10, wherein:
the belt-loop and the pair of arm-loops of the harness are a continuous length of a harness rope.

15. The back brace device of claim 10, wherein:
the T-plate is a single piece of resilient plastic material, and resilient plastic material that is bendable without breaking, and the T-plate forms a substantially rigid framework for the back brace device.
